(19) United States
(12) Patent Application Publication
(10) Pub. No.: US 2026/0176568 A1
(43) Pub. Date: Jun. 25, 2026

(54) MULTIPLEXED WELL PLATE STIMULATOR

(71) Applicants: THE JOHNS HOPKINS UNIVERSITY, Baltimore, MD (US); LOYOLA UNIVERSITY OF CHICAGO, Chicago, IL (US)

(72) Inventors: Brian Leei LIN, Baltimore, MD (US); Peter CARON, Maywood, IL (US)

(21) Appl. No.: 19/124,686

(22) PCT Filed: Nov. 1, 2023
(86) PCT No.: PCT/US2023/036589

§ 371 Date: Apr. 25, 2025

Related U.S. Application Data
(60) Provisional application No. 63/383,611, filed on Nov. 14, 2022.

(51) Int. Cl. C12M 1/42; C12M 1/00; C12M 1/32; C12N 13/00

(52) U.S. Cl. CPC C12M 35/02; C12M 23/12; C12M 23/38; C12N 13/00

(57) ABSTRACT

A well plate stimulator includes a lid configured to be coupled to a well plate comprising a plurality of wells. The well plate stimulator also includes a plurality of pairs of well conductors. Each of the pairs is configured to be inserted into a different one of the wells in the well plate when the well plate stimulator is coupled to the well plate. Each of the pairs includes a first well conductor that is coupled to a first wire and a second well conductor that is coupled to a second wire. Positive and negative portions of a signal generate an electrical current between the first and second well conductors in each pair to stimulate cells within the wells. The well conductors have an electrical conductivity that is greater than about 8×10⁶ S/m.

### CROSS-REFERENCE TO RELATED APPLICATIONS

This application is the national stage entry of International Patent Application No. PCT/US2023/036589, filed on Nov. 1, 2023, and published as WO 2024/107338 A8 on May 23, 2024, which claims the benefit of U.S. Provisional Patent Application No. 63/383,611, filed on Nov. 14, 2022, which are hereby incorporated by reference in their entireties.

### GOVERNMENT SUPPORT

This invention was made with government support under grant 1K99HL155840-01A1 awarded by NIH. The government has certain rights in the invention.

### FIELD OF THE DISCLOSURE

The present disclosure relates generally to systems and methods for stimulating cells. More particularly, the present disclosure relates to systems and methods for providing electrical stimulation to cardiac cells in a plurality of wells in a well plate.

### BACKGROUND OF THE DISCLOSURE

Electrical signals are a key component of cell-to-cell signaling in many organs. To study the cells, scientists commonly culture cells in culture plates, which can contain many wells per plate. A well plate stimulator may then be positioned on and/or coupled to the well plate. The well plate stimulator introduces an artificial electrical stimulation to the cells in the wells to simulate the electrical stimulation from the heart so that the cells may be studied.

Currently-available conventional well plate stimulators include a plurality of pairs of carbon paddles. Each pair is configured to be inserted into a different well in the well plate. The electrical current received by each pair of carbon paddles decreases proceeding away from a power source due at least in part to the pairs being connected in series as well as the electrical resistivity of the carbon paddles. In other words, the pair of carbon paddles that is farthest from the power source receives less electrical current than the pair of carbon paddles that is closest to the power source. As a result, conventional well plate stimulators only include up to six pairs of carbon paddles because additional pairs of carbon paddles would receive insufficient electrical current.

A common layout for culture plates includes up to 24 wells. When the well plate stimulator only includes six pairs of carbon paddles, the well plate stimulator is moved from quadrant to quadrant on the well plate to stimulate the cells in all 24 wells. This results in a non-sterile environment. In other words, removing carbon paddles from one quadrant of wells and then introducing them into another quadrant of the wells may introduce contaminants into the latter quadrant and also expose the plate to non-sterile environment. In addition, the carbon paddles are inflexible, can absorb nutrients and reagents added in the wells, and act as a temperature sink. Therefore, what is needed is an improved system and method for stimulating cells in a well plate in which stimulation can be applied without affecting the temperature, reagents, or sterility of the cell culture.

### SUMMARY

A well plate stimulator includes a lid configured to be coupled to a well plate comprising a plurality of wells. The well plate stimulator also includes a first wire coupled to the lid. The well plate stimulator also includes a second wire coupled to the lid. The well plate stimulator also includes a plurality of pairs of well conductors. Each of the pairs is configured to be inserted into a different one of the wells in the well plate when the well plate stimulator is coupled to the well plate. Each of the pairs includes a first well conductor that is coupled to the first wire and a second well conductor that is coupled to the second wire. A positive portion of a signal generated by a power source is transmitted through the first electrical connector and the first wire to the first well conductor. A negative portion of the signal generated by the power source is transmitted through the second electrical connector and the second wire to the second well conductor. The positive and negative portions of the signal generate an electrical current between the first and second well conductors in each pair to stimulate cells within the wells. The well conductors have an electrical conductivity that is greater than about 8×10⁶ S/m.

A system for stimulating cells is also disclosed. The system includes a well plate defining a plurality of wells. The plurality of wells includes twenty-four wells arranged in four rows and six columns. Each of the wells extends partially through the well plate from a first side of the well plate toward a second side of the well plate. Each of the wells is substantially cylindrical. Each of the wells is configured to receive a plurality of cardiac cells and a media. The system also includes a power source configured to generate a signal. The signal has a voltage from about 1 V to about 40 V and an electrical current from about 1 mA to about 120 mA. The signal includes a plurality of pulses having a frequency from about 0.5 Hz to about 2 Hz and a duration from about 12 ms to about 20 ms. The system also includes a well plate stimulator. The well plate stimulator includes a lid configured to be coupled to the first side of the well plate. The well plate stimulator also includes a first electrical connector coupled to the lid. The well plate stimulator also includes a second electrical connector coupled to the lid. The well plate stimulator also includes a first wire coupled to the lid and the first electrical connector. The first wire extends over each of the wells in the well plate when the well plate stimulator is coupled to the well plate. The well plate stimulator also includes a second wire coupled to the lid and the second electrical connector. The second wire extends over each of the wells in the well plate when the well plate stimulator is coupled to the well plate. The first and second wires are substantially parallel with one another. The well plate stimulator also includes a plurality of pairs of well conductors including at least a first pair and a twenty-fourth pair. The plurality of pairs are arranged in four rows and six columns such that each pair is configured to be inserted into a corresponding one of the wells when the well plate stimulator is coupled to the well plate. Each pair includes a first well conductor that is coupled to the first wire and a second well conductor that is coupled to the second wire. A positive portion of the signal is transmitted from the power source, through the first electrical connector and the first wire, to the first well conductor. A negative portion of the signal is transmitted from the power source, through the second electrical connector and the second wire, to the second well conductor. The well conductors have an electrical conductivity that is greater than about 3×10⁷ S/m. The electrical current in the twenty-fourth pair of the well conductors is greater than or equal to about 90% of the electrical current in the first pair of the well conductors.

A method for stimulating cells is also disclosed. The method includes introducing cells into a plurality of wells in a well plate. The plurality of wells comprises at least twenty-four wells. The method also includes introducing a media into the plurality of wells in the well plate. The method also includes placing a well plate stimulator on the well plate to seal the cells and the media within the wells. The well plate stimulator includes a plurality of pairs of well conductors. The plurality of pairs includes at least twenty-four pairs. Placing the well plate stimulator on the well plate also introduces one of the pairs of well conductors into each of the wells. The well conductors have an electrical conductivity that is greater than about 8×10⁶ S/m. The method also includes generating an electrical current in each of the wells with the pairs of well conductors to stimulate the cells in the wells. The electrical current in a pair of the electrical conductors that is farthest from a power source is greater than or equal to about 80% of the electrical current in a pair of the electrical conductors that is closest to the power source.

### BRIEF DESCRIPTION OF THE FIGURES

FIG. 1 illustrates a perspective view of a system for stimulating cells in a first (e.g., open) state, according to an embodiment.

FIG. 2 illustrates a perspective view of a portion of the system in a second (e.g., closed) state, according to an embodiment.

FIG. 3 illustrates a flowchart of a method for stimulating the cells, according to an embodiment.

### DETAILED DESCRIPTION

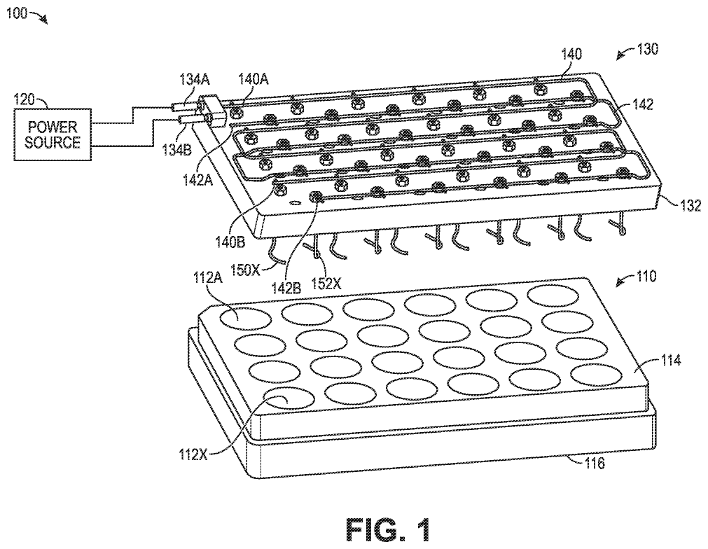

FIG. 1 illustrates a perspective view of a system 100 for stimulating cells, according to an embodiment. The system 100 may include a well plate 110. The well plate 110 may include or define a plurality of wells (twenty-four are shown: 112A-112X). However, in other embodiments, the well plate 110 may include a greater or lesser number of wells 112A-112X. In the example shown, the wells 112A-112X are arranged in a 4×6 pattern (e.g., four rows and six columns). The wells 112A-112X each extend partially through the well plate 110 from a first side 114 to a second side 116. The wells 112A-112X may each have a substantially cylindrical shape.

As described in greater detail below, one or more cells may be introduced into each well 112A-112X. The cells may be or include living cells, dead cells, or a combination thereof. The cells may be from a plant or an animal (e.g., a human). In one example, the cells may be or include cardiac cells that are removed from a heart via a biopsy. A media may also be introduced into each well 112A-112X such that the cells may be at least partially within the media. The media may be or include a liquid containing nutrients for the cells.

The system 100 may also include a power source 120. The power source 120 may be configured to generate a signal to simulate the electrical stimulation from the heart. The signal may have an electrical current from about 1 mA to about 120 mA. The signal may have a voltage from about 1 V to about 40 V. The signal may be or include a plurality of discrete pulses. The pulses may have a duration from about 8 milliseconds (ms) to about 24 ms, or about 12 ms to about 20 ms (e.g., about 16 ms). The pulses may have a frequency from about 0.2 hertz (Hz) to about 5 Hz, or about 0.5 Hz to about 2 Hz (e.g., about 1 Hz).

The system 100 may also include a well plate stimulator 130. The well plate stimulator 130 may include a lid 132. As described below, the lid 132 may be configured to be placed on and/or coupled to the first side 114 of the well plate 110 to seal the wells 112A-112X with the cells and/or media therein.

The well plate stimulator 130 may also include one or more electrical connectors (two are shown: 134A, 134B). The electrical connectors 134A, 134B may be connected to the power source 120 and/or the lid 132.

The well plate stimulator 130 may also include one or more wires (two are shown: 140, 142). The wires 140, 142 may be coupled to and/or positioned at least partially within the lid 132. The wires 140, 142 may be substantially parallel to one another. The wires 140, 142 may be arranged in a substantially serpentine and/or S-shape on/in the lid 132. This arrangement may allow the wires 140, 142 to pass over each of the wells 112A-112X in the well plate 110 when the well plate stimulator 130 is in contact with and/or coupled to the well plate 110.

The wires 140, 142 may include first (e.g., proximate) ends 140A, 142A and second (e.g., distal) ends 140B, 142B. The first ends 140A, 142A of the wires 140, 142 may be connected to the electrical connectors 134A, 134B. The first ends 140A, 142A of the wires 140, 142 may be positioned proximate to (e.g., over) the first well 112A when the well plate stimulator 130 is in contact with and/or coupled to the well plate 110, and the second ends 140B, 142B of the wires 140, 142 may be positioned proximate to (e.g., over) the twenty-fourth well 112X when the well plate stimulator 130 is in contact with and/or coupled to the well plate 110

The wires 140, 142 may be made from a material with a high electrical conductivity. More particularly, the material may have a higher electrical conductivity than carbon. For example, the material may have an electrical conductivity that is greater than about 8×10⁶ S/m, 1×10⁷ S/m, 3×10⁷ S/m, or 5×10⁷ S/m. For example, the wires 140, 142 may be made from platinum, iron, lithium, ruthenium, nickel, cobalt, zinc, tungsten, calcium, aluminum, gold, copper, silver, or a combination thereof.

The well plate stimulator 130 may also include a plurality of sets (e.g., pairs) of well conductors (twenty-four are shown 150A-150X, 152A-152X). More particularly, the well plate stimulator 130 may include a pair of well conductors corresponding to each well (e.g., well conductors 150X, 152X correspond to well 112X). The well conductors 150A-150X may be connected to and/or integral with the wire 140, and the well conductors 152A-152X may be connected to and/or integral with the wire 142.

Each pair of well conductors (e.g., well conductors 150X, 152X) may be configured to be inserted at least partially into a corresponding well (e.g., well 112X) in the well plate 110. Each well conductor (e.g., well conductors 150X, 152X) may include a first portion and a second portion. The first portions may be substantially straight. The first portions may also or instead be substantially perpendicular to the lid 132 and/or the wires 140, 142. The second portions may be curved. More particularly, the second portions may each form a portion of a circle (e.g., corresponding to the inner surface of the well 112X). For example, the second portions may form from about 10° to about 90° of a perimeter of the circle. The second portions may be separated from one another by (e.g., circumferential) gaps.

The well conductors 150A-150X, 152A-152X may be made from a material with a high electrical conductivity. More particularly, the material may have a higher electrical conductivity than carbon. For example, the material may have an electrical conductivity that is greater than about 8×10⁶ S/m, 1×10⁷ S/m, 3×10⁷ S/m, or 5×10⁷ S/m. For example, the well conductors 150A-150X, 152A-152X may be made from platinum, iron, lithium, ruthenium, nickel, cobalt, zinc, tungsten, calcium, aluminum, gold, copper, silver, or a combination thereof.

The system 100 is in a first (e.g., open) state in FIG. 1. In the open state, the well plate 110 is spaced apart from the well plate stimulator 130 (e.g., the lid 132). This may allow the cells and/or the media to be introduced into and/or removed from the wells 112A-112X.

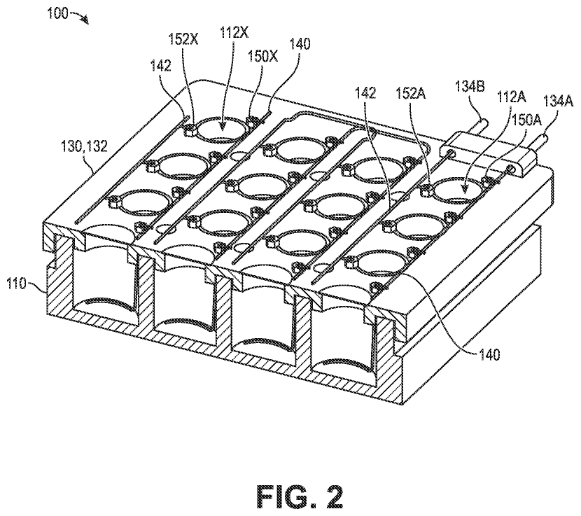

FIG. 2 illustrates a perspective view of a portion of the system 100 in a second (e.g., closed) state, according to an embodiment. In the closed state, the well plate 110 is in contact with and/or coupled to the well plate stimulator 130 (e.g., the lid 132). More particularly, in the closed state, each well 112A-112X in the well plate 110 is (e.g., hermetically) sealed by the lid 132 of the well plate stimulator 130 such that neither the cells, the media, nor any contaminants may be introduced into and/or removed from the wells 112A-112X. In addition, in the closed state, each well (e.g., well 112X) may have a corresponding pair of the well conductors 150X, 152X positioned therein.

As described in greater detail below, when the system 100 is in the closed state, the signal generated by the power source 120 may be transmitted through the electrical connectors 134A, 134B and the wires 140, 142. The wire 140 may be configured to transmit a positive portion of the signal, and the wire 142 may be configured to transmit a negative portion of the signal. The positive portion of the signal may be transmitted from the wire 140 into/through the well conductors 150A-150X, and the negative portion of the signal may be transmitted from the wire 142 into/through the well conductors 152A-152X. This may induce an electrical stimulation in each well 112A-112X that simulates the electrical stimulation from the heart. For example, an electrical current may flow between the well conductors 150X, 152X in the well 112X that stimulates the cells therein. In the embodiment shown, the system 100 may be multiplexed (e.g., a single power source may provide the signal to all well conductors).

The signal may travel from about 5 cm to about 50 cm or from about 10 cm to about 30 cm along the wire(s) 140, 142 from the (e.g., first) pair of well conductors 150A, 152A closest to the electrical connectors 134A, 134B to the (e.g., twenty-fourth) pair of well conductors 150X, 152X farthest from the electrical connectors 134A, 134B. In one embodiment, the electrical current in/between the last pair of well conductors 150X, 152X may be greater than or equal to about 70%, about 80%, or about 90% of the electrical current in/between the first pair of well conductors 150A, 152A.

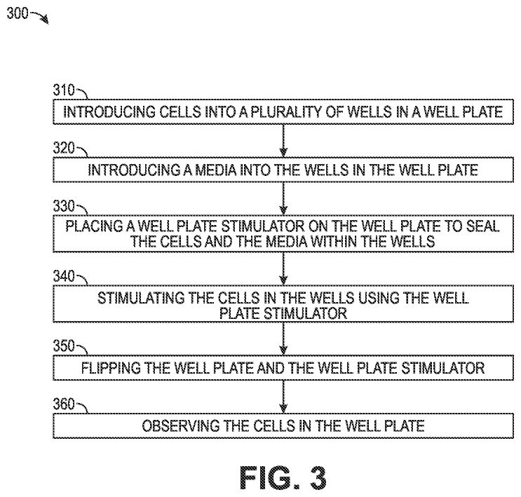

FIG. 3 illustrates a flowchart of a method 300 for stimulating the cells, according to an embodiment. An illustrative order of the method 300 is provided below; however, one or more steps of the method 300 may be performed in a different order, combined, repeated, or omitted without departing from the scope of the disclosure.

The method 300 may include introducing the cells into one or more of the wells 112A-112X in the well plate 110, as at 310. The cells may be introduced when the system 100 is in the open state.

The method 300 may also include introducing the media into one or more of the wells 112A-112X in the well plate 110, as at 320. The media may be introduced when the system 100 is in the open state.

The method 300 may also include placing the well plate stimulator 130 (e.g., the lid 132) on the well plate 110, as at 330. This switches the system 100 from the open state into the closed state. As mentioned above, switching the system 100 into the closed state (e.g., hermetically) seals the wells 112A-112X and/or positions a pair of well conductors (e.g., well conductors 150X, 152X) into each well (e.g., well 112X).

The method 300 may also include stimulating the cells in the wells 112A-112X, as at 340. As described above, this may include generating a signal with the power source 120. The signal may be transmitted through the electrical connectors 134A, 134B and the wires 140, 142 (e.g., from the first ends 140A, 142A to the second ends 140B, 142B). The positive portion of the signal may be transmitted from the wire 140 into/through the well conductors 150A-150X, and the negative portion of the signal may be transmitted from the wire 142 into/through the well conductors 152A-152X. This may induce an electrical stimulation in each well 112A-112X that simulates the electrical stimulation from the heart. For example, an electrical current may flow between the well conductors 150X, 152X in the well 112X that stimulates the cells therein.

The well conductors 150A-150X, 152A-152X may absorb less nutrients and/or reagents than conventional paddles. For example, the well conductors 150A-150X, 152A-152X may not absorb nutrients and/or reagents in the wells 112A-112X.

In one embodiment, stimulating the cells in the wells 112A-112X may minimize a drop in temperature of the cells and/or the media within the wells 112-112X at least in part due to the material (e.g., platinum) of the well conductors 150A-150X, 152A-152X. For example, a temperature of the cells and/or the media within the wells 112-112X may decrease by less than or equal to about 0.5° C., about 1° C., or about 2° C. in response to the cells being stimulated for 5 minutes with the well conductors 150A-150X, 152A-152X. In contrast, a temperature of the cells and/or the media within the wells of a conventional system may decrease by greater than or equal to about 5° C. in response to the cells being stimulated for 30 minutes at least in part due to the material (e.g., carbon) and size of the conventional paddles.

The method 300 may also include flipping the system 100, as at 350. More particularly, the well plate 110 may be on the bottom, and the well plate simulator 130 may be on the top, for steps 310, 320, 330, 340, or a combination thereof. Flipping the system 100 may cause the well plate 110 to be on the top, and the well plate stimulator 130 to be on the bottom.

The method 300 may also include observing the cells in the well plate 110, as at 360. At least a portion of the well plate 110 may be transparent. For example, the second side 116 (e.g., including the bottom of the wells 112A-112X) may be clear so that a user may observe the cells in the wells 112A-112X through the clear second side 116. In one embodiment, the user or a computing system may observe the cells through a microscope.

The system 100 may maintain a sterile environment in all (e.g., twenty-four) wells 112A-112X because there are cells and well conductors 150A-150X, 152A-152X in all of the wells 112A-112X simultaneously. As a result, the system 100 may remain in the closed state while the signal is introduced into all (e.g., twenty-four) wells 112A-112X and/or the cells are observed in all (e.g., twenty-four) wells 112A-112X.

As used herein, the terms “inner” and “outer”; “up” and “down”; “upper” and “lower”; “upward” and “downward”; “upstream” and “downstream”; “above” and “below”; “inward” and “outward”; and other like terms as used herein refer to relative positions to one another and are not intended to denote a particular direction or spatial orientation. The terms “couple,” “coupled,” “connect,” “connection,” “connected,” “in connection with,” and “connecting” refer to “in direct connection with” or “in connection with via one or more intermediate elements or members.”

The foregoing description, for purposes of explanation, used specific nomenclature to provide a thorough understanding of the disclosure. However, it will be apparent to one skilled in the art that the specific details are not required in order to practice the systems and methods described herein. The foregoing descriptions of specific examples are presented for purposes of illustration and description. They are not intended to be exhaustive of or to limit this disclosure to the precise forms described. Many modifications and variations are possible in view of the above teachings. The examples are shown and described in order to best explain the principles of this disclosure and practical applications, to thereby enable others skilled in the art to best utilize this disclosure and various examples with various modifications as are suited to the particular use contemplated. It is intended that the scope of this disclosure be defined by the claims and their equivalents below.

1. A well plate stimulator, comprising:
a lid configured to be coupled to a well plate comprising a plurality of wells;
a first wire coupled to the lid;
a second wire coupled to the lid; and
a plurality of pairs of well conductors, wherein each of the pairs is configured to be inserted into a different one of the wells in the well plate when the well plate stimulator is coupled to the well plate, wherein each of the pairs comprises a first well conductor that is coupled to the first wire and a second well conductor that is coupled to the second wire, wherein a positive portion of a signal generated by a power source is transmitted through the first electrical connector and the first wire to the first well conductor, wherein a negative portion of the signal generated by the power source is transmitted through the second electrical connector and the second wire to the second well conductor, wherein the positive and negative portions of the signal generate an electrical current between the first and second well conductors in each pair to stimulate cells within the wells, and wherein the well conductors have an electrical conductivity that is greater than about 8×10⁶ S/m.

2. The well plate stimulator of claim 1, wherein the first and second wires extend over each of the wells in the well plate when the well plate stimulator is coupled to the well plate.

3. The well plate stimulator of claim 1, wherein the first and second wires are substantially parallel with one another.

4. The well plate stimulator of claim 1, wherein the first and second wires are arranged in serpentine patterns.

5. The well plate stimulator of claim 1, wherein the plurality of pair of well conductors comprises at least twenty-four pairs arranged in series.

6. The well plate stimulator of claim 5, wherein the electrical current in a pair of the well conductors that is farthest from the power source is greater than or equal to about 80% of the electrical current in a pair of the well conductors that is closest to the power source.

7. The well plate stimulator of claim 1, wherein the first wire and the second wire also have an electrical conductivity that is greater than about 8×10⁶ S/m.

8. The well plate stimulator of claim 1, wherein the first wire, the second wire, and the plurality of pairs of well conductors comprise platinum.

9. The well plate stimulator of claim 1, wherein the plurality of pairs of well conductors are configured to stimulate cells in at least twenty-four wells simultaneously while the wells are hermetically sealed with the lid.

10. The well plate stimulator of claim 1, wherein the plurality of pairs of well conductors are configured to cause a temperature of cells within the wells to decrease by less than or equal to about 2° C. in response to the cells being stimulated for 5 minutes.

11. A system for stimulating cells, the system comprising:
a well plate defining a plurality of wells, wherein the plurality of wells comprises twenty-four wells arranged in four rows and six columns, wherein each of the wells extends partially through the well plate from a first side of the well plate toward a second side of the well plate, wherein each of the wells is substantially cylindrical, and wherein each of the wells is configured to receive a plurality of cardiac cells and a media;
a power source configured to generate a signal, wherein the signal has a voltage from about 1 V to about 40 V and an electrical current from about 1 mA to about 120 mA, wherein the signal comprises a plurality of pulses having a frequency from about 0.5 Hz to about 2 Hz and a duration from about 12 ms to about 20 ms; and
a well plate stimulator comprising:
a lid configured to be coupled to the first side of the well plate;
a first electrical connector coupled to the lid;
a second electrical connector coupled to the lid;
a first wire coupled to the lid and the first electrical connector, wherein the first wire extends over each of the wells in the well plate when the well plate stimulator is coupled to the well plate;
a second wire coupled to the lid and the second electrical connector, wherein the second wire extends over each of the wells in the well plate when the well plate stimulator is coupled to the well plate, and wherein the first and second wires are substantially parallel with one another; and
a plurality of pairs of well conductors including at least a first pair and a twenty-fourth pair, wherein the plurality of pairs are arranged in four rows and six columns such that each pair is configured to be inserted into a corresponding one of the wells when the well plate stimulator is coupled to the well plate, wherein each pair comprises a first well conductor that is coupled to the first wire and a second well conductor that is coupled to the second wire, wherein a positive portion of the signal is transmitted from the power source, through the first electrical connector and the first wire, to the first well conductor, wherein a negative portion of the signal is transmitted from the power source, through the second electrical connector and the second wire, to the second well conductor, wherein the well conductors have an electrical conductivity that is greater than about 3×10⁷ S/m, and wherein the electrical current in the twenty-fourth pair of the well conductors is greater than or equal to about 90% of the electrical current in the first pair of the well conductors.

12. The system of claim 11, wherein the first wire and the second wire also have an electrical conductivity that is greater than about 3×10⁷ S/m.

13. The system of claim 11, wherein the first wire, the second wire, and the plurality of pairs of well conductors comprise platinum.

14. The system of claim 11, wherein the cells in the twenty-four wells are stimulated simultaneously while the wells are hermetically sealed with the well plate stimulator.

15. The system of claim 11, wherein a temperature of the cells and the media within the wells decreases by less than or equal to about 1° C. in response to the cells being stimulated for 5 minutes.

16. A method for stimulating cells, comprising:
introducing cells into a plurality of wells in a well plate, wherein the plurality of wells comprises at least twenty-four wells;
introducing a media into the plurality of wells in the well plate;
placing a well plate stimulator on the well plate to seal the cells and the media within the wells, wherein the well plate stimulator comprises a plurality of pairs of well conductors, wherein the plurality of pairs comprises at least twenty-four pairs, wherein placing the well plate stimulator on the well plate also introduces one of the pairs of well conductors into each of the wells, and wherein the well conductors have an electrical conductivity that is greater than about 8×10⁶ S/m; and
generating an electrical current in each of the wells with the pairs of well conductors to stimulate the cells in the wells, wherein the electrical current in a pair of the electrical conductors that is farthest from a power source is greater than or equal to about 80% of the electrical current in a pair of the electrical conductors that is closest to the power source.

17. The method of claim 16, wherein the first wire and the second wire also have an electrical conductivity that is greater than about 8×10⁶ S/m.

18. The method of claim 16, wherein the first wire, the second wire, and the plurality of pairs of well conductors comprise platinum.

19. The method of claim 16, wherein the cells in the at least twenty-four wells are stimulated simultaneously while the wells are hermetically sealed with the well plate stimulator.

20. The method of claim 16, wherein a temperature of the cells and the media within the wells decreases by less than or equal to about 1° C. in response to the cells being stimulated for 5 minutes.
